(19) United States
(12) Patent Application Publication
(10) Pub. No.: US 2019/0111103 A1
(43) Pub. Date: Apr. 18, 2019

(54) NOVEL PEPTIDE OR SALT THEREOF, COMPOSITION FOR ENHANCING PROLIFERATION OF FIBROBLAST CELLS AND COMPOSITION FOR ENHANCING ELASTIN PRODUCTION IN FIBROBLAST CELLS AND CHONDROCYTES CONTAINING THE SAME

(71) Applicants: Hayashikane Sangyo Co., Ltd., Shimonoseki-shi (JP); Kagoshima University, Kagoshima-shi (JP)

(72) Inventors: Eri Shiratsuchi, Shimonoseki-shi (JP); Kenji Miyanari, Shimonoseki-shi (JP); Kazunari Arima, Kagoshima-shi (JP); Taihei Yamaguchi, Kagoshima-shi (JP); Takahito Komine, Kagoshima-shi (JP); Mizuki Takuno, Kagoshima-shi (JP)

(73) Assignees: Hayashikane Sangyo Co., Ltd., Shimonoseki-shi, Yamaguchi (JP); Kagoshima University, Kagoshima-shi (JP)

(21) Appl. No.: 16/155,015

(22) Filed: Oct. 9, 2018

(30) Foreign Application Priority Data
Oct. 13, 2017 (JP) 2017-199547

(51) Int. Cl. A61K 38/07; A61K 35/33; A61K 35/32; C12N 5/077; A61K 38/39

(52) U.S. Cl. CPC A61K 38/07; A61K 35/33; A61K 38/39; C12N 5/0655; C12N 5/0656; A61K 35/32

(57) ABSTRACT

A peptide or a salt thereof having an amino acid sequence represented by (a) or (b) shown below having a promotive effect on a proliferation of fibroblast cells, a composition for enhancing proliferation of fibroblast cells containing one or more of them, a composition for enhancing elastin production in fibroblast cells containing one or more peptides or salts thereof having an amino acid sequence represented by (a), (b) or (c) shown below and a composition for enhancing elastin production in chondrocytes containing one or more peptides or salts thereof having an amino acid sequence represented by (c) shown below.
(a) Val-Pro-Gly-Gly
(b) Val-Pro-Gly-Ala
(c) Val-Pro

### CROSS-REFERENCE TO RELATED APPLICATIONS

This patent application claims the benefit of foreign priority to Japanese Patent Application No. JP 2017-199547 filed on Oct. 13, 2017, the disclosure of which is hereby incorporated by reference in its entirety.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

The sequence listings disclosed in the ASCII text file submitted herewith, named “seqlist.txt” and created on Oct. 8, 2018, the size of which is 767 bytes, are hereby incorporated by reference.

### TECHNICAL FIELD

The present disclosure relates to a novel peptide having a promotive effect on a proliferation of fibroblast cells and a promoting effect on an elastin production in the fibroblast cells, a composition for enhancing proliferation of fibroblast cells and composition for enhancing elastin production in fibroblast cells and chondrocytes containing the same.

### BACKGROUND ART

Fibroblast cells, a kind of cell that constitutes a connective tissue, which is found in the connective tissues throughout body, deeply participates to formation and maintenance of elasticity of connective tissues such as skin, lung, ligament and cartilage through production of extracellular matrix substances such as collagen, elastin and hyaluronic acid, differentiation to osteoblast or cartilage cell, migration to damaged part of the tissue and the production of the extracellular matrix substances as described above.

In some cases, hypofunction of the fibroblast cells is associated with various diseases. For example, it is reported that wound healing ability of the fibroblast cells in patients of chronic obstructive pulmonary disease (COPD) is lower than that of the fibroblast cells in healthy persons (see Non-Patent Literature 1).

Elastin is a major component of elastic fiber tissue as well as collagen, which is an insoluble protein widely distributed in connective tissue of vertebrates. In organisms, large amount of elastin is found in tissues having elasticity and strechability such as bulbus arteriosus, nuchal ligament and skin, which plays various roles such as maintenance of elasticity and regulation of cell function in living bodies. Content of elastin is 50% or more by dry weight in blood vessels and nuchal ligament and about 2% in skin. It is known that elastin in living bodies is decreased or degenerated because of some factors such as ultraviolet exposure and aging and such change in the skin may cause wrinkles, sagging and loss of elasticity of skin. Lack of elasticity of the skin of the mouse that genetically lacks elastin producing ability (see Non-Patent Literature 2) and possibility of the loss of elasticity or formation of wrinkles as a result of a structural change of elastic fibers after ultraviolet exposure (see Non-Patent Literature 3). From these facts, one can expect that a substance that enhances the elastin production may contribute to the maintenance of elasticity and tension of skin and prevention and improvement of the wrinkles.

It is reported that water soluble extract of tissue of swine and horse, in particular the water soluble extract of elastin or its hydrolysate obtained from swine or horse nuchal ligament is highly useful as effective substances having biologically sound effect to skin and hair and skin conditioning effect (see Patent-Literature 1). Also, it is reported that a water soluble elastin peptide; hydrolysate of elastin obtained from fish bulbus arteriosus has a promoting effect on elastin production in skin fibroblast (see Patent-Literature 2).

### CITATION LIST

### Patent Literature

Patent Literature I: Unexamined Japanese Patent Application Kokai Publication No. 2002-205913

Patent Literature 2: Unexamined Japanese Patent Application Kokai Publication No. 2010-155820

### Non-Patent Literature

Non-Patent Literature 1: Grant-in-Aid for Scientific Research, 2010 Fiscal Year Final Research Report, “Role of lung fibroblasts on the pathogenesis of COPD”, URL https://kaken.nii.ac.jp/ja/report/KAKENHI-PROJECT-20590905/20590905seika/

Non-Patent Literature 2: Yanagisawa H. et al., “Fibulin-5 is an elastin-binding protein essential for elastic fibre development in vivo”, Nature, Nature Publishing Group (United Kingdom), Vol. 415, Issue 6868 (Jan. 10, 2002), p. 168-171

Non-Patent Literature 3: Imokawa G. et al., “Degree of Ultraviolet-Induced Tortuosity of Elastic Fibers in Rat Skin Is Age Dependent”, J. Invest. Dermatol., Nature Publishing Group (United Kingdom), Vol. 105, No. 2 (August 1995), p.254-258

### SUMMARY OF INVENTION

### Technical Problem to be Solved

However, activities of a hydrolysate of protein such as elastin greatly varies depending on some factors such as production condition since it is a mixture of a number of peptides having different amino acid sequences and molecular weights, of which composition varies depending on a condition of hydrolyzation.

Under these circumstances, as a result of intensive study on a peptide having a promotive effect on a proliferation of fibroblast cells and a promoting effect on an elastin production in the fibroblast cells made by the inventors of the present disclosure, novel peptides has been found as well as the aforementioned effects in a dipeptide known in the art. Thus, the object of the present disclosure is to provide a novel peptide having a promotive effect on a proliferation of fibroblast cells and a promoting effect on an elastin production in the fibroblast cells, a composition for enhancing proliferation of fibroblast cells and composition for enhancing elastin production in fibroblast cells and chondrocytes containing the same.

### Solution to Problem

In the first aspect of the present disclosure according to the object as described above, the aforementioned problem is solved by providing a peptide or a salt thereof having an amino acid sequence represented by (a) or (b) shown below having a promotive effect on a proliferation of fibroblast cells.

(a) Val-Pro-Gly-Gly

(b) Val-Pro-Gly-Ala

In the peptide or the salt thereof according to the first aspect of the present disclosure, the fibroblast cells may be one or both of human skin fibroblast cells and human lung fibroblast cells.

In the peptide or the salt thereof according to the first aspect of the present disclosure, the peptide or the salt thereof may also have a promoting effect on an elastin production in the fibroblast cells.

In the peptide or the salt thereof according to the first aspect of the present disclosure, the peptide or the salt thereof may also have a promoting effect on a migration of the fibroblast cells.

In the second aspect of the present disclosure, the aforementioned problem is solved by providing a composition for enhancing proliferation of fibroblast cells containing one or more peptides or salts thereof having an amino acid sequence represented by (a) or (b) shown below.

(a) Val-Pro-Gly-Gly

(b) Val-Pro-Gly-Ala

In the composition for enhancing proliferation of fibroblast cells according to the second aspect of the present disclosure, the fibroblast cells may be one or both of human skin fibroblast cells and human lung fibroblast cells.

In the third aspect of the present disclosure, the aforementioned problem is solved by providing a composition for enhancing elastin production in fibroblast cells containing one or more peptides or salts thereof having an amino acid sequence represented by (a), (b) or (c) shown below.

(a) Val-Pro-Gly-Gly

(b) Val-Pro-Gly-Ala

(c) Val-Pro

In the composition for enhancing elastin production in fibroblast cells according to the third aspect of the present disclosure, the fibroblast cells may be one or both of human skin fibroblast cells and human lung fibroblast cells.

In the fourth aspect of the present disclosure, the aforementioned problem is solved by providing a composition for enhancing elastin production in chondrocytes containing one or more peptides or salts thereof having an amino acid sequence represented by Val-Pro.

In the composition for enhancing elastin production in chondrocytes according to the fourth aspect of the present disclosure, the chondrocytes may be human chondrocytes.

### Advantageous Effects of Invention

The present disclosure provides a novel peptide having a promotive effect on a proliferation of fibroblast cells, a novel composition for enhancing proliferation of fibroblast cells, a novel composition for enhancing elastin production in fibroblast cells and a novel composition for enhancing elastin production in chondrocytes. These compositions are effective for maintenance of elasticity, prevention and improvement of dysfunction because of, for example, aging, ultraviolet exposure and disorder in skin, lung and cartilage. In addition, these compositions show fixed effect since they contain the peptide or salt thereof having established amino acid sequences.

### BRIEF DESCRIPTION OF DRAWINGS

FIG. 1 shows a graph illustrating an influence of a peptide having an amino acid sequence Val-Pro-Gly-Gly on a cell proliferation rate of normal human skin fibroblast cells.

FIG. 2 shows a graph illustrating an influence of a peptide having an amino acid sequence Val-Pro-Gly-Ala on a cell proliferation rate of normal human skin fibroblast cells.

FIG. 3 shows a graph illustrating an influence of a peptide having an amino acid sequence Val-Pro-Gly-Gly on an amount of elastin production of normal human skin fibroblast cells.

FIG. 4 shows a graph illustrating an influence of a peptide having an amino acid sequence Val-Pro-Gly-Ala on an amount of elastin production of normal human skin fibroblast cells.

FIG. 5 shows a graph illustrating an influence of a peptide having an amino acid sequence Val-Pro-Gly-Gly on cell proliferation rate of normal human lung fibroblast cells.

FIG. 6 shows a graph illustrating an influence of a peptide having an amino acid sequence Val-Pro-Gly-Ala on cell proliferation rate of normal human lung fibroblast cells.

FIG. 7 shows a graph illustrating an influence of a peptide having an amino acid sequence Val-Pro-Gly-Gly on an amount of elastin production of cartilage cells from human knee joint.

FIG. 8 shows a graph illustrating an influence of a peptide having an amino acid sequence Val-Pro-Gly-Ala on an amount of elastin production of chondrocytes from human knee joint.

FIG. 9 shows a graph illustrating an influence of a peptide having an amino acid sequence Val-Pro on an amount of elastin production of chondrocytes from human knee joint.

### EMBODIMENT OF THE INVENTION

A peptide or salt thereof according to the first embodiment of the present disclosure has an amino acid sequence represented by (a) or (b) shown below and has a promotive effect on a proliferation of fibroblast cells.

(a) Val-Pro-Gly-Gly

(b) Val-Pro-Gly-Ala

The peptide and salt thereof having the amino acid sequence represented by (a) or (b) shown above have a promotive effect on a proliferation of fibroblast cells. Example of fibroblast cells includes skin fibroblast cells, lung fibroblast cells and ligament fibroblast cells.

The peptide and salt thereof having the amino acid sequence represented by (a) or (b) shown above also have a promoting effect on an elastin production in the fibroblast cells. In addition, peptide and salt thereof having the amino acid sequence represented by (a) or (b) shown above have a promoting effect on a migration of the fibroblast cells.

Moreover, the peptide and salt thereof having the amino acid sequence represented by (a) or (b) shown above as well as a peptide and salt thereof having the amino acid sequence represented by (c) shown below have a promoting effect on an elastin production in chondrocytes.

(c) Val-Pro

It is already known that the peptide having the amino acid sequence represented by (c) shown above has an inhibitory effect to angiotensin conversion enzyme (see Unexamined Japanese Patent Application Kokai Publication No. 2007-182424). The promoting effect on the elastin production in the chondrocytes of that peptide has been found by the inventors of the present disclosure for the first time.

The production method of the peptides having the amino acid sequence represented by (a), (b) and (c) shown above is not particularly limited and any methods such as hydrolysis of protein, organic chemical synthesis, gene engineering synthesis and fermentation.

One or both of N-terminus amino group and C-terminus carboxylic group in the peptide having the amino acid sequence represented by (a), (b) and (c) shown above may be in the form of salt (amine salt and/or carboxylate salt). The kind of salt is not particularly limited and they may be in the form of any salts as far as they do not inhibit various effects to be described below and compatible with pharmaceutical or food use. Example of amine salt includes chloride, bromide, sulfate, hydrogen sulfate, nitrate, carbonate, hydrogen carbonate, phosphate, hydrogen phosphate, dihydrogen phosphate, acetate, propionate, lactate, citrate, tartrate. Example of carboxylate salt includes alkali salt such as sodium salt and potassium salt; alkali earth salt such as calcium salt and magnesium salt and ammonium salt. In case that both of the N-terminus amino group and the C-terminus carboxylic group form salt, the salt may be intramolecular salt (Zwitter ionic salt).

One or more peptides or salts thereof selected from the group consisting of the peptide or salt thereof having the amino acid sequence represented by (a) or (b) shown above may be used as one or more of an agent for promoting proliferation of fibroblast cells, an agent for promoting elastin production of fibroblast cells and an agent for promoting immigration of fibroblast cells. One or more peptides or salts thereof selected from the group consisting of the peptide or salt thereof having the amino acid sequence represented by (a), (b) or (c) shown above may be used as an agent for promoting elastin production of chondrocytes.

By mixing one or more peptides or salts thereof selected from the group consisting of the peptide or salt thereof having the amino acid sequence represented by (a) or (b) shown above with carriers and the like, the composition may be used as a pharmaceutical composition having one or more of effect of promoting proliferation of fibroblast cells, effect of promoting elastin production of fibroblast cells and effect of promoting immigration of fibroblast cells. Also, by mixing one or more peptides or salts thereof selected from the group consisting of the peptide or salt thereof having the amino acid sequence represented by (a), (b) or (c) shown above with carriers and the like, the composition may be used as a pharmaceutical composition having effect of promoting proliferation of fibroblast chondrocytes.

Administration form of the medical composition to human or animals includes oral, rectal, parenteral (for example, intravenous, intramuscular, subcutaneous, etc.). Although the dosage is to be adjusted appropriately depending on dosage forms of the pharmaceutical compositions, method of administration, purpose of administration and the age, weight and condition of the administration subject and it is difficult to determine unambiguously, in humans, generally the amount of active ingredient contained in the formulation is preferably 1 day 0.1 to 2000 mg/kg adult human. Naturally, the dosage can vary depending on various factors and in some cases smaller dose than the dosage as mentioned above may be sufficient, or in other case the dosage exceeding the range as mentioned above may be required.

When the composition is formulated in an oral dosage form, it may be formulated in the form of tablets, granules, powders, capsules, coatings, solutions, suspension and the like, and when the composition is formulated in parenteral dosage form, it may be formulated in the form of injections, infusions, suppositories and the like. Any known method may be used for formulation. For example, the peptide or salt thereof having the amino acid sequence represented by (a), (b) or (c) shown above may be admixed with pharmaceutically acceptable carrier or diluent, stabilizers and other desired additives to formulate in the desired dosage form as described above.

One or more peptides or salts thereof selected from the group consisting of the peptide or salt thereof having the amino acid sequence represented by (a) or (b) shown above may be used as a food or a supplement having one or more of effect of promoting proliferation of fibroblast cells, effect of promoting elastin production of fibroblast cells and effect of promoting immigration of fibroblast cells by formulated into food products directly or adding to another food products or formulating in the form of capsules, tablets or in any form usually used for food or supplements. Also, one or more peptides or salts thereof selected from the group consisting of the peptide or salt thereof having the amino acid sequence represented by (a), (b) or (c) shown above may be used as a food or a supplement having one or more of effect of promoting elastin production of chondrocytes by formulated into food products directly or adding to another food products or formulating in the form of capsules, tablets or in any form usually used for food or supplements.

When the peptide or salt thereof having the amino acid sequence represented by (a), (b) or (c) shown above is ingested or administered by admixing with food products, it optionally may be mixed with excipients, fillers, binders, thickeners, emulsifiers, colorants, flavors, food additives, flavorings and the like and the food product for certain application may be formulated in the form of powders, granules, tablets and the like. Also, peptide or salt thereof having the amino acid sequence represented by (a), (b) or (c) shown above may be ingested as the supplement product having the effect as described above by mixing with raw materials for foodstuff to formulate into food product.

One or more peptides or salts thereof selected from the group consisting of the peptide or salt thereof having the amino acid sequence represented by (a) or (b) shown above may be used as a feed or a functional feed having one or more of effect of promoting proliferation of fibroblast cells, effect of promoting elastin production of fibroblast cells and effect of promoting immigration of fibroblast cells by formulated into the feed directly or adding to the feed. Also, one or more peptides or salts thereof selected from the group consisting of the peptide or salt thereof having the amino acid sequence represented by (a), (b) or (c) shown above may be used as a feed or a functional feed having one or more of effect of promoting elastin production of chondrocytes by formulated into the feed directly or adding to the feed.

For administrating the peptide or salt thereof having the amino acid sequence represented by (a), (b) or (c) shown above to animals such as live stocks, it may be formulated into a functional feed by mixing in the feed of the raw material in advance. It is also possible to administer the peptide or salt thereof having the amino acid sequence represented by (a), (b) or (c) shown above by adding to the feed. In other words, the feed containing the peptide or salt thereof having the amino acid sequence represented by (a), (b) or (c) shown above as an active ingredient may be used as functional feed that is safe and has an effects as described above of livestock such as pigs, cows, horses and sheep, etc., fish and pets (dogs, cats, birds) by adding to the feed for these animals.

### EXAMPLES

Examples are provided below for confirmation of the effect of use of the present disclosure. In the following examples, “a peptide having an amino acid sequence represented by X-Y-Z” is also written as “a peptide X-Y-Z”. In graphs showing experimental results, amino acids are denoted using one-letter code instead of three-letter code.

In the following examples, three peptides Val-Pro-Gly-Gly, Val-Pro-Gly-Ala and Val-Pro used are purchased from Kokusan Chemical Co., Ltd. In controlled experiments, hydrolysate of elastin from bonitos bulbus arteriosus used is obtained from Hayashikane Sangyo Co., Ltd.

### Example 1

### Proliferation Test of Normal Human Skin Fibroblast Cells

Skin fibroblast cells cultured in a culture flask was seeded in a 96-well plate at 3,500 cells/cm² (100 4/well, medium: 10% FBS DMEM). After incubating for 24 hours at 37° C. under 5% CO₂ condition, the liquid medium in the wells was removed, replaced with DMEM containing 0.5% FBS and incubated for 24 hours under similar conditions. The liquid medium in the wells was removed and the wells were washed with DPBS, after which DPBS was removed. Solutions of predetermined concentrations of peptide Val-Pro-Gly-Gly (VPGG) and peptide Val-Pro-Gly-Ala (VPGA) were prepared using DMEM containing 0.5% FBS were prepared and 100 μL of the solution was added to each well. Fibroblast cells were incubated for 4 days at 37° C. under 5% CO₂ condition and supernatant was recovered. The supernatant recovered was used for a measurement of amount of elastin production in Example 2. DMEM containing 0.5% FBS was added and cells were counted using cell counting kit-8 (10 μL/well) and a cell proliferation rate relative to the cell number obtained from the sample without adding the peptides.

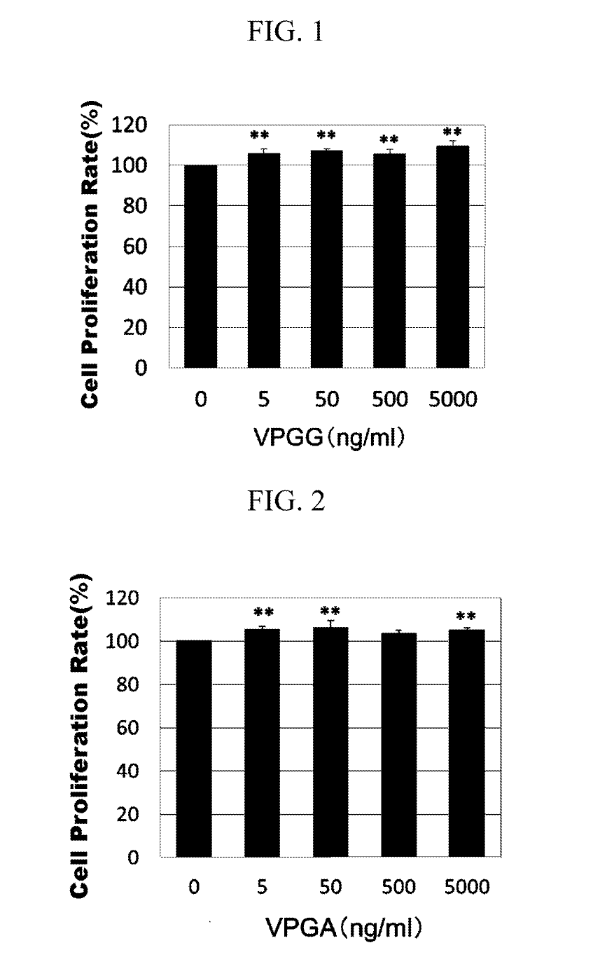

Results are shown in FIG. 1 and FIG. 2. An error bar shows standard deviation. Symbol “**” means a significant difference of p<0.01 in Dunnett's test. In both of the peptide Val-Pro-Gly-Gly and Val-Pro-Gly-Ala, it was found that addition of 5 ng/mL or more of the peptide resulted in significant increase of the cell proliferation rate; in other words, an effect for promoting cell proliferation was found. Similar experiment was carried out by using a hydrolysate of elastin from bonitos bulbus arteriosus. The peptide Val-Pro-Gly-Gly and peptide Val-Pro-Gly-Ala showed higher cell proliferation rate than that of the hydrolysate of elastin from bonitos bulbus arteriosus.

### Example 2

### Elastin Production Test in Normal Human Skin Fibroblast Cells

Quantitative measurement of the amount of elastin production in normal human skin fibroblast cells was carried out using ELISA method. Sample solutions for calibration curve were prepared using solubilized elastin (from bovine nuchal ligament) as standard by diluting a stock solution to concentrations of 2000, 1000, 500, 250, 125, 62.5, 31.25 and 0 ng/mL by DMEM containing 0.5% FBS. The sample solutions thus prepared were added to Corning 96 well EIA/RIA Half Area Flat Bottom Plate (High binding) by 40 μL each and incubated at 4° C. for 1.5 hours to be fixed on the plate. The supernatant collected in Example 1 were also fixed on the plate similarly by adding the supernatant onto the plate by 40 μL each. The liquid in the wells were discarded and wells were washed with PBST and blocked with a blocking buffer containing 0.1% gelatin (150 μL) for 1 hour. After the wells were washed with PBST, 40 μL of primary antibody (rabbit anti-elastin antibody: Anti-tropo elastin (Rabbit-Poly) PR385) diluted by 500 times was added to each well and incubated for 1 hour. After the wells were washed with PBST, 40 μL of secondary antibody (HRP conjugate goat anti-rabbit antibody: Peroxidase-Labeled Affinity Purified Antibody To Rabbit IgG(H+L)) diluted by 1000 times was added to each well and incubated for 30 minutes. After the liquid in the wells was discarded and wells were washed with PBST, 40 mL of substrate solution (TMB Peroxidase substrate:Peroxidase Substrate Solution B=1:1 solution) was added to each well and incubated at room temperature for 5 minutes. Enzymatic reaction was quenched by adding 40 μL of 1N H₂SO₄, absorption at 450 nm (A₄₅₀) was measured, from which the amount of elastin production was estimated using the calibration curve and the amount of elastin production per cell was calculated by dividing the result by the number of the cells obtained in Example 1.

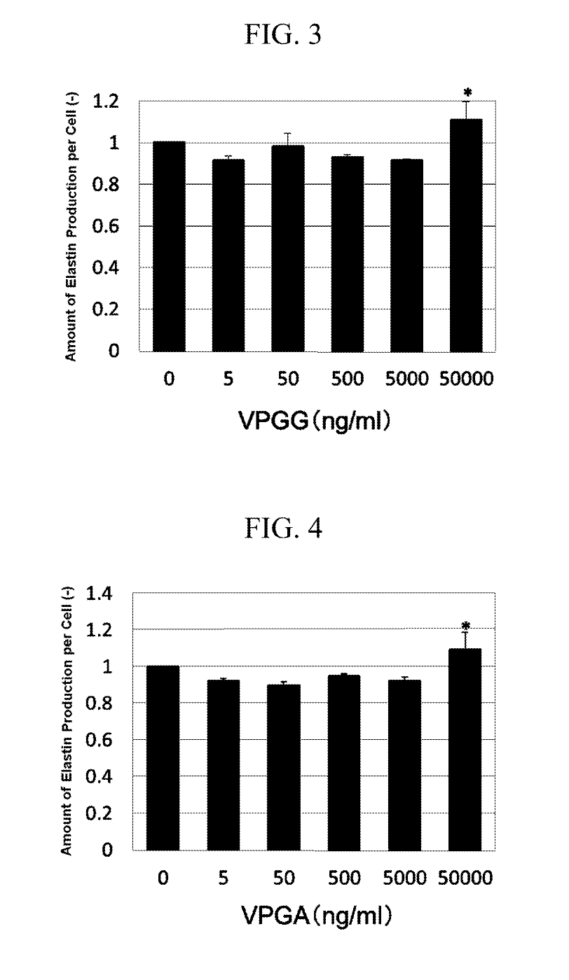

Results are shown in FIG. 3 and FIG. 4. An error bar shows standard deviation. Symbol “*” means a significant difference of p<0.05 in Dunnett's test. In both of the peptide Val-Pro-Gly-Gly and Val-Pro-Gly-Ala, it was found that addition of 50,000 ng/mL or more of the peptide resulted in significant increase of the amount of elastin production; in other words, an effect for promoting elastin production in normal human skin fibroblast cells was found.

Similar experiment carried out using normal human lung fibroblast cells showed that in both of the peptide Val-Pro-Gly-Gly and Val-Pro-Gly-Ala, it was found that addition of 50000 ng/mL or more of the peptide resulted in slight effect of promoting elastin production.

### Example 3

### Proliferation Test of Normal Human Lung Fibroblast Cells

Lung fibroblast cells cultured in a culture flask was seeded in a 96-well plate at 2,500 cells/cm² (100 μL/well, medium: 10% FBS :DMEM). After incubating for 24 hours at 37° C. under 5% CO₂ condition, the liquid medium in the wells was removed, replaced with DMEM containing 0.5% FBS and incubated for 24 hours under similar conditions. The liquid medium in the wells was removed and wells were washed with DPBS, after which DPBS was removed. Solutions of predetermined concentrations of peptide Val-Pro-Gly-Gly (VPGG) and peptide Val-Pro-Gly-Ala (VPGA) were prepared using DMEM containing 0.5% FBS were prepared and 100 μL of the solution was added to each well. Fibroblast cells were incubated for 3 days at 37° C. under 5% CO₂ condition and supernatant was recovered. The supernatant recovered was used for a measurement of amount of elastin production in Example 4. DMEM containing 0.5% FBS was added and cells were counted using cell counting kit-8 (10 μL/well).

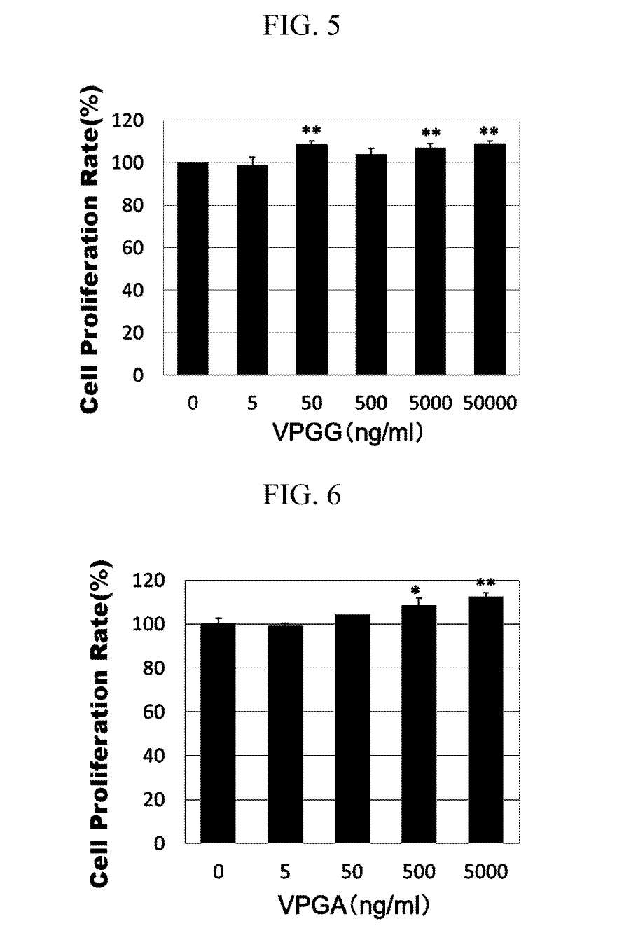

Results are shown in FIG. 5 and FIG. 6. An error bar shows standard deviation. Symbols “*” and “**” mean significant difference of p<0.05 and p<0.01 in Dunnett's test, respectively. In both of the peptide Val-Pro-Gly-Gly and Val-Pro-Gly-Ala, it was found that addition of 5 ng/mL or more of the peptide resulted in significant increase of the cell proliferation rate; in other words, an effect for promoting cell proliferation was found.

### Example 4

### Migration Activity Test of Normal Human Skin Fibroblast Cells

Effect of peptide Val-Pro-Gly-Gly on a migration activity of fibroblast cells that relates to processes of recovery and regeneration of tissue and wound healing was investigated. To bottom of each well of 24-well plate of CytoSelect (registered trade mark) 24-well Cell Migration Assay Kit (Cell Biotics), DMEM medium (control), 500 μL of peptide Val-Pro-Gly-Gly or peptide Val-Pro-Gly (positive control: see “Elastin—Structure, Function and Pathology”, edited and written by Hiroyuki Ito, Elastin Study Group of Japan (not for sale), p. 124-137, 2008) dissolved in DMEM medium were added. Then, 300 μL of cell suspension of human skin fibroblast cells (P4 to 5) prepared by dispersing the cell in DMEM media (1×10⁶ cells/mL) was added inside membrane inserts (pore size: 8 μm). The inserts were inserted in wells and incubated in a cell incubator at 37° C. for 20 hours (in preliminary examination, duration of incubation was also set to 6 hours). After the reaction, the medium was withdrawn from the inserts and non-migrated cells that did not penetrate through the membrane was removed using cotton swab (both sides of 2 swabs/well). Cells were stained by adding 400 μL of cell stain liquid and staining for 10 minutes at room temperature. Then the inserts were washed with distilled water, dried and stain was confirmed by optical microscopic observation. Then the inserts were moved to empty wells, to which 200 mL of extracting liquid was added and reacted by stirring at room temperature for 10 minutes. 100 mL of reaction mixture was placed in 96-well plate and absorbance at 560 nm was measured using a plate reader.

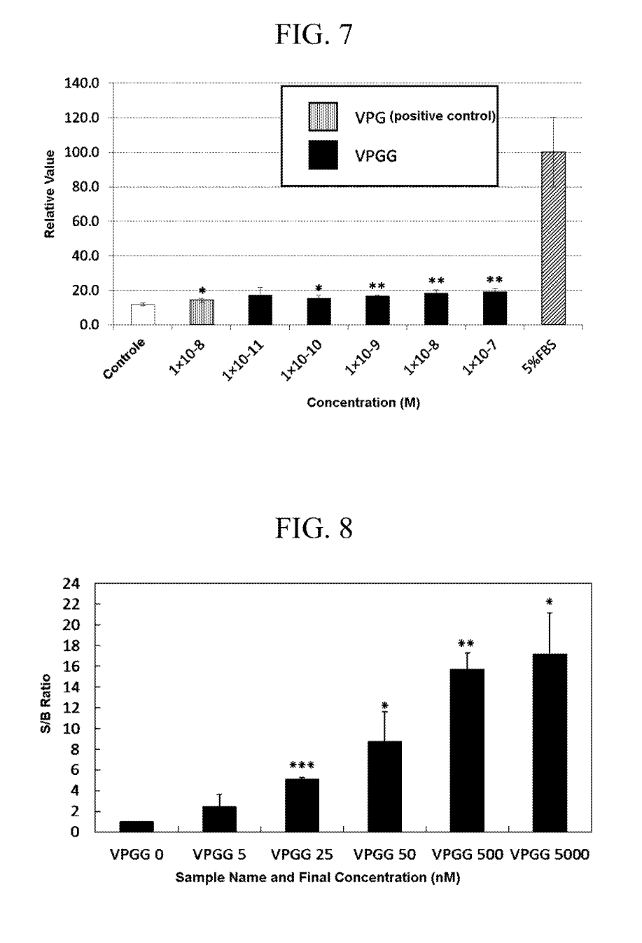

Result is shown in FIG. 7. An error bar shows standard deviation. Symbols “*” and “**” mean significant difference of p<0.05 and p<0.01 in Dunnett's test, respectively. The peptide Val-Pro-Gly-Gly shows significantly higher effect for promoting cell migration than that of the peptide Val-Pro-Gly which is known to have the effect for promoting cell migration.

### Example 5

### Elastin Production Test in Normal Human Knee Joint Chondrocytes

Normal human knee joint chondrocytes (NHAc-kn: 1.0×10⁵ cells/mL) were seeded in a 24-well plate (900 μL/well, medium: CBM+0.5% FBS) and incubated under humidification condition at 37° C. in 5% CO₂ atmosphere for 48 hours. The medium was exchanged and peptide VPGG and peptide VP was added (100 μL/well, final concentration: 0 to 5000 nM) and incubated under humidification condition at 37° C. in 5% CO₂ atmosphere for 48 hours. From the cell culture thus obtained, supernatant was collected and dot-blotted onto a PVDF membrane. After blocking with PBST containing 5% skim milk, the membrane was washed with PBST. The membrane was reacted with rabbit anti-elastin antibody as a primary antibody, washed with PBST and reacted with HRP conjugate goat anti-elastin antibody as a secondary antibody and washed with PBST. The membrane was reacted with a detection reagent ImmunoStar (FUJIFILM Wako Pure Chemical Corporation) and the amount of elastin production was estimated based on intensities of signals obtained.

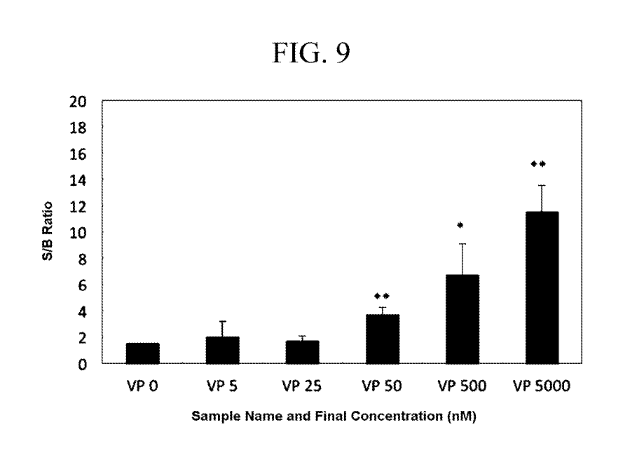

Results are shown in FIG. 8 and FIG. 9. An error bar shows standard deviation. Symbols “*”, “**” and “***” mean significant difference of p<0.05, p<0.01 and p<0.001 in Dunnett's test, respectively. In both of the peptide Val-Pro-Gly-Gly and Val-Pro-Gly-Ala, it was found that addition of 25 nM or 50 nM or more of the peptide resulted in significant increase of the amount of elastin production; in other words, an effect for promoting elastin production in normal human skin fibroblast cells was found.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limited sense, and the scope of the invention is defined only by the included claims, along with the full range of equivalents to which such claims are entitled.

The present application claims the benefit of Japanese application No. 2017-199547, filed Oct. 13, 2017, the entitle disclosure of which is incorporated herein by reference herein.

1. A peptide or a salt thereof having an amino acid sequence represented by (a) or (b) shown below having a promotive effect on a proliferation of fibroblast cells.
(a) Val-Pro-Gly-Gly
(b) Val-Pro-Gly-Ala

2. The peptide or the salt thereof according to claim 1, wherein the fibroblast cells are one or both of human skin fibroblast cells and human lung fibroblast cells.

3. The peptide or the salt thereof according to claim 1, wherein the peptide or the salt thereof also have a promoting effect on an elastin production in the fibroblast cells.

4. The peptide or the salt thereof according to claim 1, wherein the peptide or the salt thereof also have a promoting effect on a migration of the fibroblast cells.

5. A composition for enhancing proliferation of fibroblast cells containing one or more peptides or salts thereof having an amino acid sequence represented by (a) or (b) shown below.
(a) Val-Pro-Gly-Gly
(b) Val-Pro-Gly-Ala

6. The composition for enhancing proliferation of fibroblast cells according to claim 5, wherein the fibroblast cells are one or both of human skin fibroblast cells and human lung fibroblast cells.

7. A composition for enhancing elastin production in fibroblast cells containing one or more peptides or salts thereof having an amino acid sequence represented by (a), (b) or (c) shown below.
(a) Val-Pro-Gly-Gly
(b) Val-Pro-Gly-Ala
(c) Val-Pro

8. The composition for enhancing elastin production in fibroblast cells according to claim 7, wherein the fibroblast cells are one or both of human skin fibroblast cells and human lung fibroblast cells.

9. A composition for enhancing elastin production in chondrocytes containing one or more peptides or salts thereof having an amino acid sequence represented by Val-Pro.

10. The composition for enhancing elastin production in chondrocytes according to claim 9, wherein the chondrocytes are human chondrocytes.


SEQUENCE LISTING (2 sequences)

SEQ ID NO: 1 - protein, 4 aa, Artificial Sequence
  a novel tetrapeptide
VPGG

SEQ ID NO: 2 - protein, 4 aa, Artificial Sequence
  a novel tetrapeptide
VPGA